(19) United States
(12) Patent Application Publication
(10) Pub. No.: US 2007/0128252 A1
(43) Pub. Date: Jun. 7, 2007

(54) COMPOSITIONS AND METHODS FOR TREATMENT OF BODY WEIGHT CONDITIONS

(75) Inventors: Loren Spencer Ward, Twin Falls, ID (US); Eric D. Bastian, Twin Falls, ID (US); Starla J. Paulsen, Twin Falls, ID (US); Colum P. Dunne, Kilkenny (IE)

Correspondence Address: BANNER & WITCOFF, LTD., TEN SOUTH WACKER DRIVE, SUITE 3000, CHICAGO, IL, 60606, US

(73) Assignee: Glanbia Nutritionals (Ireland) Limited, Kilkenny (IE)

(21) Appl. No.: 11/553,817

(22) Filed: Oct. 27, 2006

Related U.S. Application Data
(63) Continuation-in-part of application No. 10/969,468, filed on Oct. 20, 2004.
Application No. 10/969,468 is a continuation-in-part of application No. 10/371,534, filed on Feb. 21, 2003.
(60) Provisional application No. 60/360,709, filed on Mar. 1, 2002.
(60) Provisional application No. 60/730,748, filed on Oct. 27, 2005.

(51) Int. Cl. A61K 38/17; A61K 47/00; A61K 35/20

(52) U.S. Cl. 424/439; 514/2; 424/535

(57) ABSTRACT

A nutritional supplement composition having a therapeutically effective amount of peptides is provided for the treatment of body weight conditions. The nutritional supplement may also comprise milk minerals, such as calcium and/or be combined with a protein source including κ-casein fragment 106-169. The nutritional supplement composition is administered in amounts effective for limiting weight gain and/or enhancing weight loss, as well as promoting overall good health, in the treatment of body weight conditions, including overweight and obesity. In one embodiment, the supplement is administered through a calorically restricted diet.

### CROSS REFERENCE TO RELATED APPLICATIONS

This application is a continuation-in-part of U.S. patent application Ser. No. 10/969,468, filed Oct. 20, 2004, which is a continuation-in-part of U.S. patent application Ser. No. 10/371,534, filed on Feb. 21, 2003, which claims the benefit of U.S. Provisional Application No. 60/360,709, filed on Mar. 1, 2002. This application also claims the benefit of U.S. Provisional Application No. 60/730,748, filed on Oct. 27, 2005. The disclosures of these applications are incorporated herein by reference.

### BACKGROUND OF THE INVENTION

The present invention is directed to compositions and methods for treatment of body weight conditions by administering a therapeutically effective nutritional supplement composition. More particularly, the nutritional composition, which includes a milk mineral blend and protein components, is effective for enhancing weight loss and/or limiting weight gain.

Obesity is a significant worldwide health concern that affects both young and old. In the United States, it is estimated that more than 50% of men and women are overweight. The degree to which a person may be overweight can be evaluated based on the person's “body mass index” or “BMI”, which is calculated as follows:
BMI=Weight in kilograms (kg)/[Height in meters (m)]².

In 2000, almost 20% of the population fell into the obese category as defined by a BMI of greater than or equal to 30. Problems associated with obesity include cardiovascular disease, diabetes mellitus, certain types of cancer, osteoarthritis and sleeping disorders. Obesity and related disorders account for almost 10% of US health care expenditures.

Over 90% of body energy is stored in adipose (fat) tissue. Adipose tissue has a number of important functions, the most obvious of which is to “buffer” the daily influx of dietary fat entering the blood circulation and release fatty acids as a source of metabolic fuel when needed. Other functions include the production and release of adipsin (essential for blood clotting), angiotensinogen (involved in blood pressure control), and leptin (a hormone involved in energy control). An accumulation of adipose tissue leads to overweight and obesity.

Diet is known to have effects on weight control. Excesses in diet, such as high caloric intake and consumption of high fat foods, can result in undesired weight gain and poor health. Similarly, a diet lacking one or more nutrients also can have a negative impact on weight control and health. For example, literature suggests that that a diet deficient in calcium can contribute to the occurrence obesity. Shi et al., “Effects of dietary calcium on adipocyte lipid metabolism and body weight regulation in energy-restricted aP2-agouti transgenic mice” FASEB J 15(2), 291-93 (2001); Zemel et al., “Regulation of adiposity by dietary calcium,” FASB J 14(9) 1132-38 (2000).

Mechanisms in the human body also are known to impact weight gain. For example, when food is consumed, the body releases a peptide, cholecystokinin (CCK), which acts to signal satiety as a result of promoting secretion of enzymes and other bodily fluids and other physical reaction within the gastric system. It has been shown that CCK release results in appetite reduction so that the person will stop eating. Proteins such as κ-casein fragment 106-169, also referred to as glycomacropeptide (GMP), are known to stimulate the release of CCK.

Various weight control compositions and methods for use by overweight and obese adults are known. Typically, however, the methods focus on a particular weight control mechanism in order to control weight gain or promote weight loss, with little or no regard for providing a balanced diet. For example, U.S. Pat. No. 6,384,087 to Zemel discloses methods and materials for treating or avoiding obesity in humans and other animals. The patent discloses that the obesity-control benefits can be achieved by providing a diet high in calcium. Additionally, the patent teaches that individuals are maintained on a restricted caloric diet. The weight control mechanism of this patent is directed to providing increased levels of one specific nutrient, calcium, while optionally limiting caloric intake, without promoting a balanced diet. As another example, U.S. Pat. No. 6,207,638 to Portman discloses a nutritional intervention composition for enhancing and extending satiety by stimulating the release of CCK. The composition includes a protein, a glycomacropeptide, long chain fatty acids, calcium (in the form of calcium carbonate or calcium lactate) and a combination of soluble and insoluble fibers. The patent teaches that the composition can be taken orally to permit a person to be satiated with a lower calorie intake. The weight management mechanism of this composition is directed to limiting caloric intake, without addressing overall nutritional requirements.

There remains a need for methods of improving human diets in order to maintain an ideal weight, reduce weight gain and/or enhance weight loss while promoting a nutritionally balanced diet. There also remains a need for dietary compositions to provide essential nutrients associated with a healthy diet to reduce incidence of overweight and obesity and maintain overall health.

### SUMMARY OF THE INVENTION

The present invention is directed to compositions for the management of body weight and treatment of body weight conditions, such as overweight or obesity. According to one aspect of the invention, a weight control composition is provided that includes one or more of a milk mineral blend in an amount effective for decreasing adiposity, a protein compound in an amount effective for enhancing satiety after consumption of food, and enzyme-inhibiting peptides in an amount effective for controlling fat metabolism. The milk mineral blend includes calcium, and the protein compound includes κ-casein fragment 106-169. According to another aspect of the invention, a nutritional supplement for maintaining and/or reducing weight is provided. The supplement can include a milk mineral composition having calcium in an amount effective for decreasing adiposity. The supplement also can include a protein composition having K-casein fragment 106-169 in an amount effective for enhancing satiety to limit or curtail consumption of food. The supplement also can include enzyme-inhibiting peptides, such as angiotensin converting enzyme- inhibiting peptides, in an amount effective for enhancing weight loss in treating an overweight condition or obesity.

According to yet another aspect of the invention, methods of maintaining a desired weight and treating an overweight condition or obesity include administering to an individual in need of such treatment a nutritional composition limiting weight gain and/or enhancing weight loss, as well as promoting overall good health. The composition can include therapeutically effective amounts of a milk mineral component, a protein component and/or an enzyme-inhibiting peptide component. The composition can be taken directly by an individual or administered via a food product fortified with the composition. The amount of nutritional supplement composition when administered via a food product can be suitably selected, for example, such that a daily serving or a predetermined number of servings of the food product delivers an amount of the composition effective for maintaining a desired weight or treating an overweight condition or obesity. The composition can be administered just prior to or after consumption of food, as part of a meal or as a snack between meals.

The nutritional supplement composition, generally in the form of a powder of appropriate particle size, can be incorporated into a wide variety of types of food products. By way of example, the nutritional composition can be added to acidic juice beverages (e.g., orange juice, apple juice, grape juice, grapefruit juice, cranberry juice, or blended juices), acidic beverages (e.g., sport beverages, GATORADE®), neutral pH beverages (e.g., milk UHT dairy, RTD nutritional, soy milk, or shakes and other blended beverages such as milkshakes, smoothies, frappes), nutritional supplement foodstuffs (e.g., high-energy protein bars), confectionery products (e.g., high calcium chews, chewing gum, chocolate, or cookies), dairy products (e.g., yogurt, ice cream, milk, cheese, processed cheese, or butter), and farinaceous products (e.g., bread, muffins, biscuits, cereal or rolls). Alternatively, the nutritional supplement composition can be administered directly, such as in the form of tablets or capsules and optionally combined with other minerals and/or vitamins.

### DESCRIPTION OF THE DRAWINGS

The foregoing and other features and advantages of the present invention will be more fully understood from the following detailed description of illustrative embodiments taken in conjunction with the accompanying drawings in which:

FIG. 1 is a graph depicting the change in weight for each treatment group in the weight reduction phase of a study conducted with compositions having milk derived protein products in accordance with various embodiments of the invention.

FIGS. 2 and 3 are graphs depicting the reduction in body weight in mice provided milk-derived peptides in accordance with various embodiments of the invention compared with control treatment groups.

FIG. 4 is a graph depicting the lean-to-fat ratio of mice provided milk-derived protein isolates in accordance with an embodiment of the invention compared with control treatment groups.

FIGS. 5 and 5a are graphs depicting the changes in lean tissue mass and fat mass, respectively, in mice provided various diets in accordance with various embodiments of the invention.

FIGS. 6 and 6a are graphs depicting bone mineral density of mice over the study period and at the end of the study in accordance with various embodiments of the invention.

FIGS. 7 and 7a are graphs depicting bone mineral content of mice over the study period and at the end of the study in accordance with various embodiments of the invention.

### DETAILED DESCRIPTION OF THE INVENTION

The present invention is directed to compositions and methods for maintaining a predetermined body weight range and treating an overweight condition or obesity by enhancing weight loss and/or limiting weight gain and promoting good health. Overweight and obesity has been associated to some degree with inadequate intake of dairy products, and more particularly the minerals present in dairy products. It has been discovered that an overweight condition and obesity can be effectively treated by administering nutritional supplement compositions, either directly or via food products fortified with the compositions, in accordance with the practice of the present invention. The nutritional supplement compositions contain therapeutically effective amounts of milk mineral, protein and enzyme-inhibiting peptides and are administered prior to or during a meal. The nutritional supplement compositions also can be administered to an individual seeking to maintain a desired body weight.

Treatment can be enhanced by use of additional ingredients in the composition to address other mechanisms for weight control. The compositions can include κ-casein fragment 106-169 (or a source of such peptides) to limit caloric intake by providing a sense of satiety, which will lead to termination of eating. Additionally, enzyme-inhibiting peptides may be included to assist with regulation of adiposity by controlling fat metabolism.

The terms “treat,” “treating,” “treatment,” and similar terms as used herein refer to the administration of the nutritional supplement compositions to individuals, particularly humans, who are overweight or obese, for alleviating, suppressing, inhibiting, or otherwise reducing the extent to which the individual is overweight or obese or any symptom associated therewith. The terms “treat,” “treating,” “treatment,” and similar terms also are used herein to refer to the prophylactic administration of the nutritional supplement compositions to individuals who may be at risk of, or otherwise wish to avoid, becoming overweight or obese.

One component of the nutritional supplement composition is milk minerals. The term “milk mineral,” as used herein, refers to a mineral complex obtained from whey or milk. The mineral complex contains a balanced form of calcium, copper, magnesium, phosphorus, potassium, selenium and zinc. Milk mineral has a relatively neutral taste, in contrast to the chalky taste of calcium carbonate. Whey fractions that are high in calcium have been demonstrated to exhibit higher calcium bioavailability than are exhibited by calcium carbonate and calcium lactate. Ranhotra et al., “Bioavailability of Calcium in a High Calcium Whey Fraction,” Nutrition Research, Vol. 17 Nos. 11-12, pp. 1663-1670 (1997). For optimal absorption, calcium and phosphorous preferably are present in a calcium-to-phosphorous ratio of about 1:1 to 2:1, e.g., a ratio similar to that found in both milk and in bone. The milk mineral also typically contains quantities of lactose and bioactive proteins. Milk mineral is also commonly referred to as “milk calcium.”

Milk mineral provides various benefits as compared to supplements having other forms of calcium. Calcium supplements and calcium-fortified foods contain calcium in such forms as calcium carbonate, calcium lactate, calcium citrate, calcium chloride, and calcium hydroxide. These forms of calcium, however, can yield undesirable flavors and/or can strip desirable aroma and flavor compounds from food products. Use of milk minerals can avoid these problems. More significantly, the milk mineral complex delivers not only calcium but a balanced and pure form of the other milk minerals, including calcium, copper, magnesium, phosphorus, potassium, selenium and zinc, that are present only in milk and dairy products and that are important to a healthy diet. As a result, the milk mineral complex provides a balanced form of minerals, including calcium that is a preferred form of calcium and other minerals from a nutritional standpoint.

Suitable methods of obtaining milk mineral by extraction from whey or milk are known to persons skilled in the art. One suitable extraction method is described in U.S. Pat. No. 5,639,501, the disclosure of which hereby is incorporated by reference in its entirety. Additionally, commercially available milk mineral products include TRUCAL® products, which are commercially available from Glanbia Nutritionals, Inc. of Monroe, Wis. A typical composition of milk mineral is illustrated in Table 1 below.

TABLE 1
Typical Composition for Milk Mineral Powder

| Component | Relative Amount (% by weight) |
|---|---|
| Total Minerals | 50-90% |
| Inorganic Mineral (Ash) | 45-85% |
| Organic Mineral (Citrate) | 1-10% |
| Calcium | 15-35% |
| Magnesium | 0-10% |
| Phosphorous | 7-15% |
| Potassium | 0-5% |
| Zinc | 0-1% |
| Lactose | 0-15% |
| Protein | 1-15% |
| Free Moisture | 2-5% |
| Fat | 0-5% |

One important attribute of milk mineral is the calcium-to-magnesium ratio.

Predetermined calcium-to-magnesium ratios are desired to limit or avoid leaching of other important minerals, which in turn may lead to bone brittleness and can even increase the risk of osteoporosis. Without wishing to be bound by any theories, high dietary Ca:Mg ratios interfere with magnesium absorption because calcium and magnesium share common intestinal absorption pathways. When calcium levels are high with respect to magnesium levels, calcium competes with magnesium for the absorption pathways, resulting in hypomagnesaemia (low magnesium in the blood).

The natural milk minerals, especially calcium, copper, magnesium, phosphorus, potassium, selenium and zinc, are of great importance in nutrition. Calcium, for example, is essential to many body functions, such as muscle function regulation, blood clotting, hormone regulation, nerve function, and enzyme activation. Calcium in milk mineral has a high bioavailability, which is enhanced by vitamin D, lactose, gastrointestinal acidity, and certain fibers. Also, the balanced form of calcium, copper, magnesium, phosphorus, potassium, selenium and zinc, and vitamin D in milk mineral helps to minimize calcium depletion through urinary loss.

The balance of minerals and bioactive proteins in the milk mineral renders food products fortified with the compositions of the present invention effective for healthy weight maintenance and in the treatment of the conditions of overweight and obesity. While not wanting to be bound by any theory, the following provides a discussion of the various mechanisms by which body weight conditions can be treated using the compositions of the present invention.

The milk minerals of the present compositions provide high calcium bioavailability effective for managing body weight and treating conditions including overweight and obesity. A common metabolic defect in cellular calcium ion handling is thought to contribute to the occurrence of weight gain and obesity. Low calcium intake increases intracellular calcium concentration in the adipocyte (fat cell) thereby switching its metabolism from lipolysis (fat breakdown) to lipogenesis (fat synthesis) and fat accumulation. By increasing the amount of calcium intake, intracellular calcium concentration is reduced, which leads to increased lipolysis and decreased lipogenesis.

Thus, it is desired to provide a daily intake of an amount of calcium effective for weight maintenance and/or loss through reduction of fat tissue mass.

In another aspect of the present invention, the nutritional supplement composition includes a protein source such as whey proteins or other suitable food protein. Whey proteins occur in milk as soluble, globular proteins. Generally, they are an important source of protein needed for overall good health and nutrition. The primary proteins and peptide constituents derived from whey proteins include α-lactalbumin and β-lactoglobulin, κ-casein fragment 106-109, lactoferrin, bovine serum albumin, lactoperoxidase, and immunoglobulins.

An important peptide constituent is κ-casein fragment 106-109. These peptides function as an appetite suppressant by stimulating the release of the gastrointestinal hormone CCK. CCK is effective for short-term control of eating behavior because it generates responses in the body that are associated with satiety, thereby resulting in termination of the meal. Thus, by administering an effective amount of κ-casein fragment 106-169 prior to, during, or even shortly after a meal, the amount of food eaten during a meal can be limited while providing a sense of satiety. Additionally, administering κ-casein fragment 106-169 between meals when a person may feel hungry may also provide a sense of satiety, thereby avoiding undesired snacking between meals.

Sources of κ-casein fragment 106-169 include PROVON® 190 and PROVON® 290, which are commercially available from Glanbia Nutritionals, Inc. of Monroe, Wis.

Suitable methods for producing κ-casein fragment 106-169 are known to those of skill in the art and are, for example, described in U.S. Pat. Nos. 5,278,288 and 5,280,107, incorporated herein by reference in their entirety, which describe processes for producing κ-casein fragment 106-169 from milk raw materials such as cheese whey and whey protein concentrates.

In another aspect of the present invention, the composition includes enzyme-inhibiting peptides. Sources of such peptides include casein, whey proteins, soy proteins, or any other suitable commercially available food protein which can be processed according to any methods known to those of skill in the art to provide the peptides. One example of such peptides are those that inhibit angiotensin converting enzyme (ACE). Angiotensin II is a hormone that is synthesized and secreted by adipose cells. Literature has shown that angiotensin II may be involved in control of adiposity through regulation of lipid synthesis and storage of adipocytes. Some dairy peptides are associated with the inhibition of angiotensin converting enzyme (ACE). Thus, by administering a therapeutically effective amount of ACE-inhibiting peptides, weight loss can be enhanced. Another type of enzyme-inhibiting peptides are those that will inhibit the enzymatic breakdown of CCK. One or more enzyme-inhibiting peptides can be included in the composition.

To obtain the compositions of the present invention, the components selected for the compositions can be processed as desired prior to preparation of the nutritional supplement compositions. Milk mineral extract typically is purified, spray dried, and ground into a powder having an appropriate particle size to permit mixing with a liquid or solid food product if desired. The milk mineral extract has calcium and other minerals as shown in Table 1. Similarly, the protein component typically is purified, dried, and ground into a powder. The protein component has κ-casein fragment 106-169 and, if desired, amino acids and other nutrients essential for overall good health and nutrition. ACE-inhibiting peptides also can be provided in the nutritional supplement composition. The desired components, which are selected from milk mineral extract, protein component and ACE-inhibiting peptides and combinations thereof, are blended to provide the nutritional supplement compositions of the present invention. The composition optionally can include other ingredients, such as minerals, vitamins, flavorings and colorants, in accordance with techniques well known to persons skilled in the art.

Suitable particle sizes for the composition will depend on such factors as the physical properties (e.g., liquid or solid, specific gravity, pH, viscosity, etc.) of the food product into which the powder is mixed. The mean particle size most often ranges from about 0.1 microns to about 300 microns, more usually from about 1 micron to about 100 microns. For neutral pH beverages, such as milk, a more finely ground powder preferably is employed so that a suspension of the powder can be easily formed. Because the solubility of the powder increases as pH decreases, less finely ground powders typically can be used, for example, in acidic juice beverages and in acidic beverages, in which the powder solubilizes.

The nutritional supplement composition in powder form can be used as an additive for a wide variety of types of food products, including acidic juice beverages (e.g., orange juice, apple juice, grape juice, grapefruit juice, cranberry juice, or blended juices), acidic beverages (e.g., sport beverages, GATORADE®), neutral pH beverages (e.g., milk UHT dairy, RTD nutritional, soy milk, or shakes and other blended beverages such as milkshakes, smoothies, frappes), nutritional supplement foodstuffs (e.g., high-energy protein bars), confectionery products (e.g., high calcium chews, chewing gum, chocolate, or cookies), dairy products (e.g., yogurt, ice cream, milk, cheese, processed cheese, or butter), and farinaceous products (e.g., bread, muffins, biscuits, cereal or rolls). The relative amount by weight of the nutritional supplement compositions combined with a food product depends on such factors as the density and the serving size of the food product. Typically, the amount of nutritional supplement compositions ranges from 0.1 to about 10 percent by weight, based on the total weight of the food product.

Alternatively, the nutritional supplement composition can be prepared in a form to be directly administered to an individual. By way of example, the composition can be prepared in the form of tablets, chewable tablets, capsules, and liquid syrup.

The formulation of the composition and, if administered via a food product, the amount of the composition blended into the food product, are selected to provide desired amounts of the particular components so as to be effective for controlling weight gain and/or weight loss. By way of example, a typical nutritional supplement composition may be administered to provide between at least about 0.5 and about 6 grams or more of calcium, between at least about 0 and about 10 grams or more of κ-casein fragment 106-169, and between at least about 0 to about 20 grams or more of ACE-inhibiting peptides per serving of the composition. The amount of composition administered can be adjusted as desired to account for differences in physical characteristics and nutritional requirements of the individuals to whom the composition is administered.

In accordance with the methods of the present invention, body weight conditions, including overweight and obesity, are effectively managed and treated. That is, an individual of healthy condition and having a generally ideal weight can manage his weight and maintain a desired weight range. An individual who has a weight in excess of a desired range, and may be considered overweight or obese, can be effectively treated by limiting weight gain and/or promoting weight loss. A therapeutically effective amount of the nutritional supplement composition is administered to an individual to provide these benefits.

### EXAMPLES

The following examples further illustrate preferred embodiments of the present invention but are not be construed as in any way limiting the scope of the present invention as set forth in the appended claims.

### Example 1

This example illustrates preparation of a beverage fortified with a nutritional supplement composition. The components were mixed to yield a product having the composition set forth in Table 2.

TABLE 2

| Ingredient | Amount (weight %) | Weight (grams) |
|---|---|---|
| Water | 70.03 | 350.17 |
| PROVON ® 190 | 10.71 | 53.55 |
| Crystalline fructose | 7.00 | 35.00 |
| TRUCAL ® FP D7 | 1.48 | 7.38 |
| Carrageenan | 0.08 | 0.40 |
| Maltodextrin | 10.00 | 50.00 |
| Flavor | 0.70 | 3.50 |
| Color | 0.001 | 0.005 |

The liquid ingredients and carrageenan were mixed on high speed for about 5 minutes for hydration. The remaining dry ingredients were blended together and added slowly to the liquid mixture and mixed on low speed for between about 5 to about 10 minutes.

An 11-ounce serving of the fortified drink provides 1 gram of calcium, 30 grams protein, 6 grams of κ-casein fragment 106-169, and 1 gram of ACE-inhibiting peptide components.

### Example 2

This example illustrates a formulation for a first dry beverage mix fortified with a nutritional supplement composition. The components were mixed to yield a product having the composition set forth in Table 3.

TABLE 3

| Ingredient | Amount (weight %) | Weight (grams) |
|---|---|---|
| PROVON ® 190* | 45 | 18.5 |
| TRUCAL ®* | 10 | 4 |
| Protein Component/ACE-inhibiting | 10 | 4 |
| peptide | | |
| Fructose | 31 | 13 |
| Carrageenan | <1 | 0.3 |
| Dispersion Aids | <1 | 0.3 |
| Flavor | 3 | 1.4 |
| Color | <1 | <0.1 |

*Available from Glanbia Nutritionals Inc., of Monroe, Wisconsin

The dry mix is prepared to be ready for mixing with a sufficient volume of a desired liquid, such as water or skim milk, to provide a beverage.

A single serving (41.5 grams) of the dry mix delivers about 127 calories, about 20 grams of protein, about 1 gram of calcium, about 4 grams of κ-casein fragment 106-169, and about 2 grams of ACE-inhibiting peptide components.

### Example 3

This example illustrates a formulation for a second dry beverage mix fortified with a nutritional supplement composition. The components were mixed to yield a product having the composition set forth in Table 4.

TABLE 4

| Ingredient | Amount (weight %) | Weight (grams) |
|---|---|---|
| PROVON ® 190* | 45 | 18.5 |
| TRUCAL ®* | 10 | 4 |
| Protein Component/ACE-inhibiting | 10 | 4 |
| peptide source | | |
| Fructose | 31 | 13 |
| Carrageenan | <1 | 0.3 |
| Dispersion Aids | <1 | 0.3 |
| Flavor | 4 | 1.8 |
| Color | <1 | <0.1 |

*Available from Glanbia Nutritionals, Inc., of Monroe, Wisconsin.

The dry mix is prepared to be ready for mixing with a sufficient volume of a desired liquid, such as water or skim milk, to provide a beverage.

A single serving (41.5 grams) of the dry mix delivers about 125 calories, about 20 grams of protein, about 1 gram of calcium, about 4 grams of κ-casein fragment 106-169, and about 4 grams of ACE-inhibiting peptide components.

### Example 4

This example illustrates a formulation for a third dry beverage mix fortified with a nutritional supplement composition. The components were mixed to yield a product having the composition set forth in Table 5.

TABLE 5

| Ingredient | Amount (weight %) | Weight (grams) |
|---|---|---|
| Prolibra* | 54 | 27 |
| Fructose | 27 | 13.3 |
| Maltodextrin | 5 | 2.6 |
| Carrageenan | 1 | 0.5 |
| Flavor | 13 | 6.6 |

*Available from Glanbia Nutritionals, Inc., of Monroe, Wisconsin

The dry mix is prepared to be ready for mixing with a sufficient volume of a desired liquid, such as water or skim milk, to provide a beverage.

A single serving (50 grams) of the dry mix delivers about 162 calories, about 21 grams of protein, about 1 gram of calcium, about 4 grams of κ-casein fragment 106-169, and about 4 grams of ACE-inhibiting peptide components.

### Example 5

This example describes a randomized, placebo-controlled study that evaluated the regulation of metabolism and weight loss following the administration of an effective amount of milk-derived nutritional ingredients. Mice were chosen as the preferred species for evaluating the effects of high fat diet due to the genetic similarity to humans and the fact that they respond to high fat diets and develop obesity in a way that is very similar to humans. For example, in one study (Petro et al., “Fat, Carbohydrate, and Calories in the Development of Diabetes and Obesity in the C57BL/6J Mouse,” Metabolism, Vol. 53, No. 4, pp. 454-57, 2004), mice had significant increases in weight, glucose, and insulin after diet-induced obesity, similar to what would be expected for humans.

Procedures

1. Weight Gaining Phase

One hundred and twenty (120) mice were initially fed a high-fat diet in order to cause them to gain excessive weight. With excessive weight gain, the mice could serve as a model to evaluate the extent to which the exemplary milk derived components will trigger weight loss. The mice utilized in the study were evaluated to ensure that they were not “diet resistant,” such that they would remain lean even when fed a high fat diet ad lib. Individual animals, including mice, may differ in their responses to high-fat diet and are therefore not expected to develop obesity with the same severity and at the same rate. One study (Burcelin et al., Am. J Physiol. Endocrinol. Metab. 282: E834-E842, 2002) suggests that only 50-80% of the mice utilized in these studies become obese. In another study, mouse weights started at 14-16 grams and lean mice gained about 8 grams while obese mice gained about 12 grams (Petro et al., 2004). Due to this variation in mouse weights, the trial was divided into two phases: 1) a weight gaining phase and 2) a weight reduction phase.

During the weight gaining phase, one hundred twenty (120) C57BL/6J mice (60 females, 60 males) were enrolled in the study at five weeks of age and were fed a high fat diet for eight weeks. To ensure an adequate number of mice became obese, a sample size of 120 was chosen to ensure that 80 mice would be obese following eight weeks on the high fat diet. The trial was designed based on the prediction that sample sizes of 3-4 mice of each sex per group would have 95% power (overall ANOVA p<0.01) to show differences among groups with similar ranges of variance.

Upon initiation of the study, all 120 mice were scanned using dual-energy x-ray absorptiometry (DEXA) scanned and provided a high-fat diet given ad lib, wherein fat comprised about 60% of the kilocalories. The diet utilized was D12492, commercially available from Research Diets, Inc., New Brunswick, N.J. The mice were then weighed once per week for the next eight weeks on this high fat diet. After eight weeks, the heaviest obese mice for the weight reduction phase of this trial were chosen. These mice were weight-matched across treatment groups for the trial before starting the weight reduction phase.

2. Weight Reduction Phase

The weight reduction phase was intended to evaluate the effectiveness of compositions manufactured in accordance with select embodiments of the invention in reducing both fat and overall weight. More specifically, the weight reduction phase examined the effect of different components of such compositions and dosages of those components to separately evaluate the effectiveness of each component for reducing overall weight and amount of fat with respect to the mice.

The 80 heaviest mice were again DEXA scanned and divided into ten weight-matched treatment groups of eight mice each (4 females and 4 males in each group). As seen in Table 6, each treatment group was provided a different diet to determine the effect on weight reduction phase from varying dosages of calcium, whey derived peptides and whey derived protein. A more complete description of each treatment group is as follows.

Group 1 represented the baseline obese mice. These mice were immediately sacrificed and the needed tissue and serum samples were collected for later analysis. Samples of interscapular brown adipose tissue, epididymal fat, and renal fat were collected from the mice (collectively “total fat”). The tissues were weighed and individually snap frozen in liquid nitrogen.

Group 2 represented an obese control group. Mice in this group were continued on the high-fat diet (ad lib) provided during the weight gaining phase of the study.

Group 3 represented a high protein, low fat base diet to serve as a control for a high protein, low fat standard diet. The base diet, provided ad lib, comprised about 30% protein, about 35% carbohydrates, and about 35% fat when measured by the amount of total kilocalories supplied. The diet was further fortified with about 0.4% calcium. Group 4 represented a high protein, low fat base diet of Group 3 with restricted energy intake. Mice in this group were switched to the base diet with energy intake restricted to 70% of calories on the high fat diet. The energy restriction for this group was calculated by utilizing the weekly food intake data from the last two weeks of the weight gaining phase of the study The average amount of food consumed per cage per day was considered 100% intake, and 70% of this amount was fed to the 70% energy restricted groups. This method was also employed to determine 30% energy restriction for Groups 5-8.

Groups 5 and 6 represented a base diet fortified with calcium. Mice in Group 5 were provided the 70% base diet, however, the calcium content was increased to 1.2% using a milk mineral calcium source, TruCalD50 (1.2% TruCalD50), commercially available from Glanbia Nutritionals of Monroe, Wis. Group 6 represented a base diet fortified with even more calcium. Mice in this group were provided the 70% base diet, however, the calcium content was increased to 2.4% using TruCalD50 (2.4% TruCalD50).

Group 7 and 8 represented a base diet with at least a portion of the protein being replaced by milk-derived peptides, including ACE peptides. Mice in Group 7 were provided the 70% base diet with 50% protein replaced with ACE hydrolysate (100% ACE hydrolysate). Group 8 represented a base diet with 100% of the protein being replaced with peptides, including ACE peptides, from milk derived products. Mice in this group were provided the 70% base diet with 100% protein replaced with ACE hydrolysate (100% ACE hydrolysate).

Group 9 represented a base diet with at least a portion of the protein in the form of whey protein isolates, including κ-casein fragment 106-169 (or GMP). Mice in this group were fed the 70% base diet with 50% of the protein replaced with PROVON® (50% PROVON®), commercially available from Glanbia Nutritionals of Monroe, Wis. Group 10 represented a base diet with 100% of the protein in the form of whey protein isolates. Mice in this group were fed the 70% base diet with 100% of the protein replaced with PROVON® (100% PROVON®).

TABLE 6

| | | Caloric | | | | | Whey | Intact |
|---|---|---|---|---|---|---|---|---|
| | | Intake | Fat | | Carb | Fortified | Peptides | Whey |
| Group | (n) | (%) | (%) | Protein (%) | (%) | Ca (%) | (%) | Protein (%) |
| 1 | 8 | — | — | — | — | — | — | — |
| 2 | 8 | 100 | 60 | 20 | 20 | 0 | 0 | 0 |
| 3 | 8 | 100 | 35 | 30 | 35 | 0 | 0 | 0 |
| 4 | 8 | 70 | 35 | 30 | 35 | 0 | 0 | 0 |
| 5 | 8 | 70 | 35 | 30 | 35 | 1.2 | 0 | 0 |
| 6 | 8 | 70 | 35 | 30 | 35 | 2.4 | 0 | 0 |
| 7 | 8 | 70 | 35 | 30 | 35 | 0 | 50 | 0 |
| 8 | 8 | 70 | 35 | 30 | 35 | 0 | 100 | 0 |
| 9 | 8 | 70 | 35 | 30 | 35 | 0 | 0 | 50 |
| 10 | 8 | 70 | 35 | 30 | 35 | 0 | 0 | 100 |

The 72 mice in Groups 2-10 were fed the prescribed diets for six weeks. Individual mouse body weights and food intakes per cage were measured once per week during this 6-week period. After six weeks, all mice were DEXA scanned.

3. Statistical Analysis

Two-way analysis of covariance (ANCOVA) was used for groups 2-10 (with descriptive statistics), followed by Fisher's least significant difference t-test (t-LSD) post-hoc between group comparisons. Repeated measures ANCOVA (RMANCOVA) was used to analyze the group differences in body weight across all seven time points. The seven mean weight measurements during weight loss were (repeated) dependent measures, with group (Groups 2-10) as the grouping factor, and “weight at the start of diet” as an adjustment covariate. The “diet start weight” covariate was essential to control for individual differences in weight at diet start.

Results

1. Body Weights

All mice gained weight during the weight gaining phase of the study. After six weeks, the 80 mice selected for the weight reducing phase were significantly heavier than those mice that were not selected. In general, male mice exhibited greater weight gain than female mice.

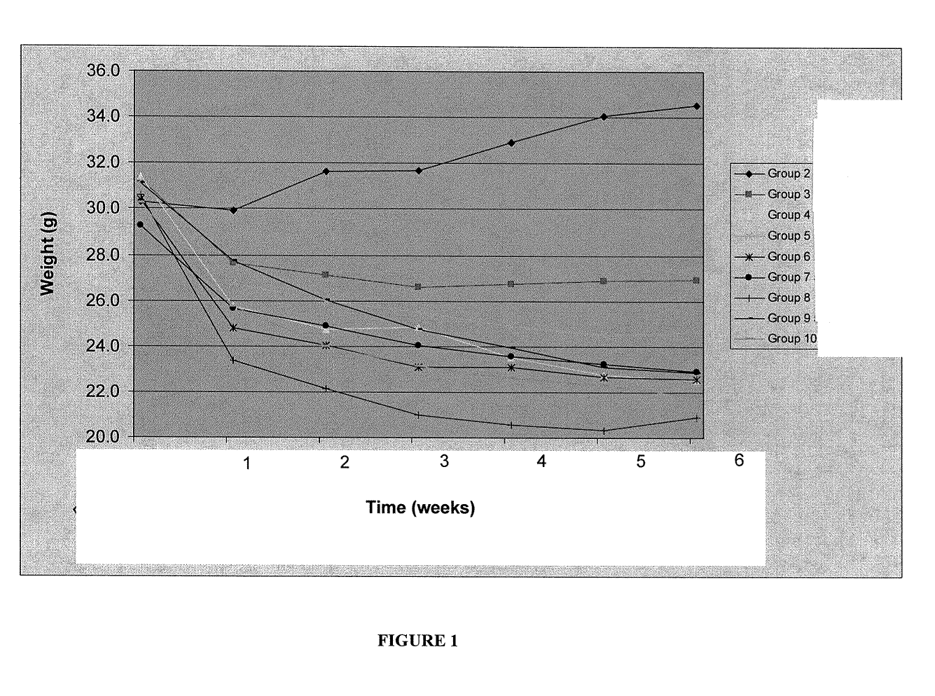

FIG. 1 shows the body weights over time for mice in Groups 2-10 during weight reduction. None of the groups were significantly different at the start of weight reduction (baseline date May 26-2005; p>0.05); however, the overall group effect over time was highly significant. As seen in FIG. 1, mice on the high-fat diet (Group 2) continued to gain weight throughout the trial.

In contrast, mice on caloric-restricted diets formulated with the experimental compositions exhibited rapid, dramatic weight loss. Specifically, Groups 2 (high fat) and 3 (base diet ad lib) were significantly different from the other weight loss groups (Groups 4-10 with 70% calorie restriction). Notably, Group 8 (0% protein, 100% peptides) had the lowest weight over all time points except baseline.

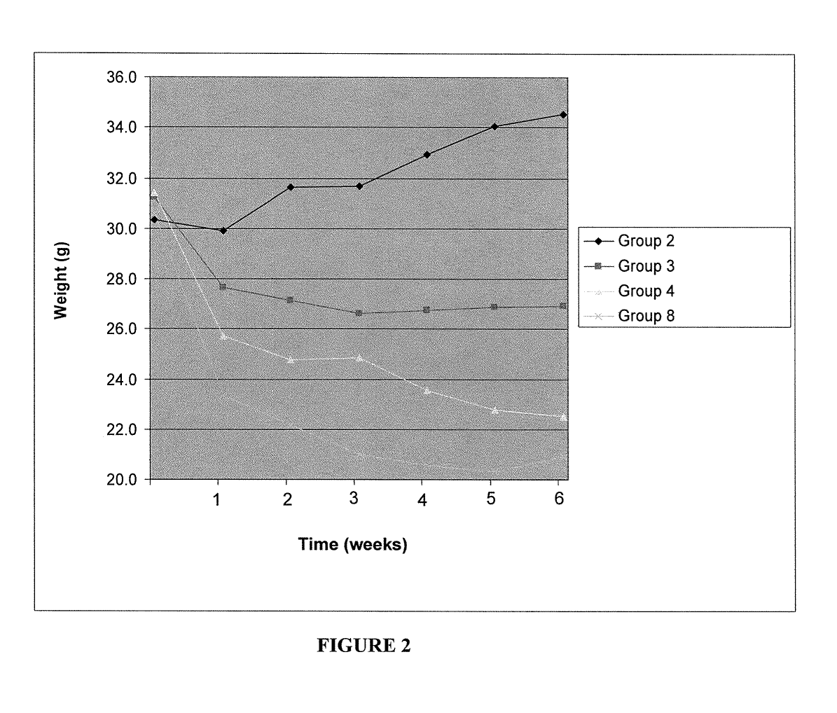

FIG. 2 illustrates the reduction in body weight in the mice provided experimental diets with 100% of the protein replaced by peptides from milk derived products (Group 8).

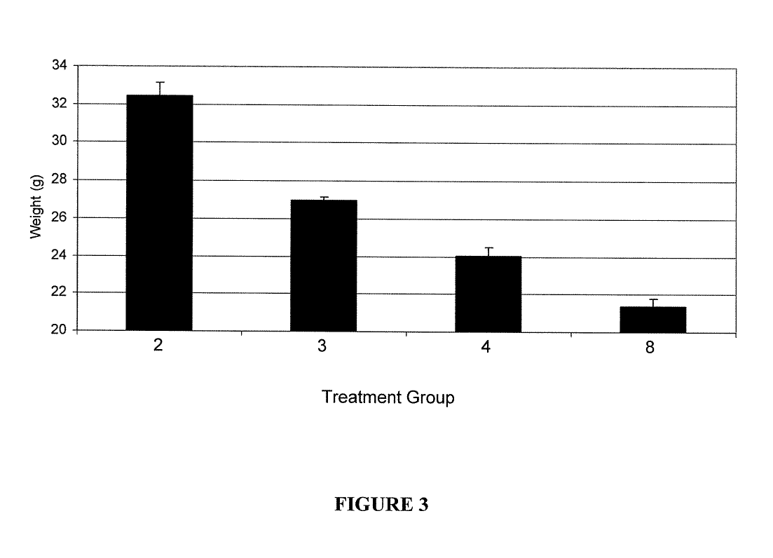

The figure shows the body weights over time for Groups 2, 3, 4 and 8 during the weight reducing phase of the study. The differences over time between Groups 2, 3, 4, and 8 were significant (RMANCOVA p<0.05). In addition, comparisons (using LSD tests) showed that the weights in Groups 4 and 8 were significantly different (p<0.05). Indeed, the amount of peptide supplied had a dramatic impact on the rate of body weight reduction. FIG. 3 shows the unweighted means of body weights of Groups 2, 3, 4, and 8 throughout the weight reduction phase of the study.

As readily seen in FIG. 3, Group 8 (fed a calorically restricted diet having 100% of the protein replaced by peptides from milk-derived products) was drastically lower than both the base diet (Group 3) and the calorically restricted base diet (Group 4). With reference to FIG. 1, the mice in Group 8 exhibited the highest decrease in weight during the initial weeks of the study. By the third week, these mice were approximately 4 grams lighter in weight than any other treatment group.

2. Lean Tissue and Fat Mass

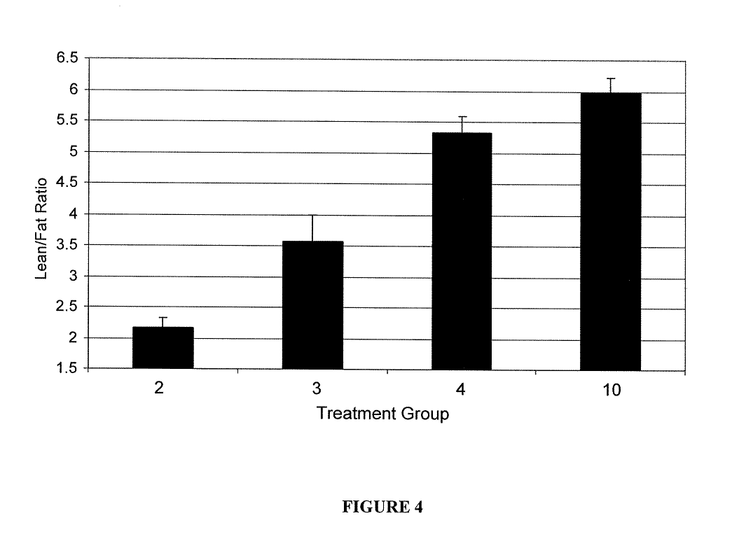

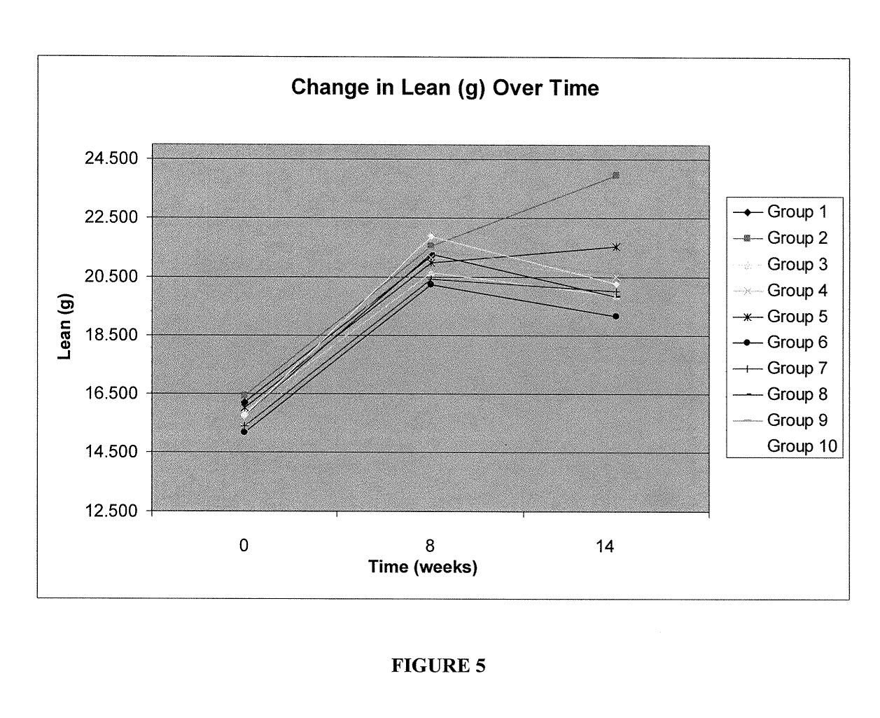

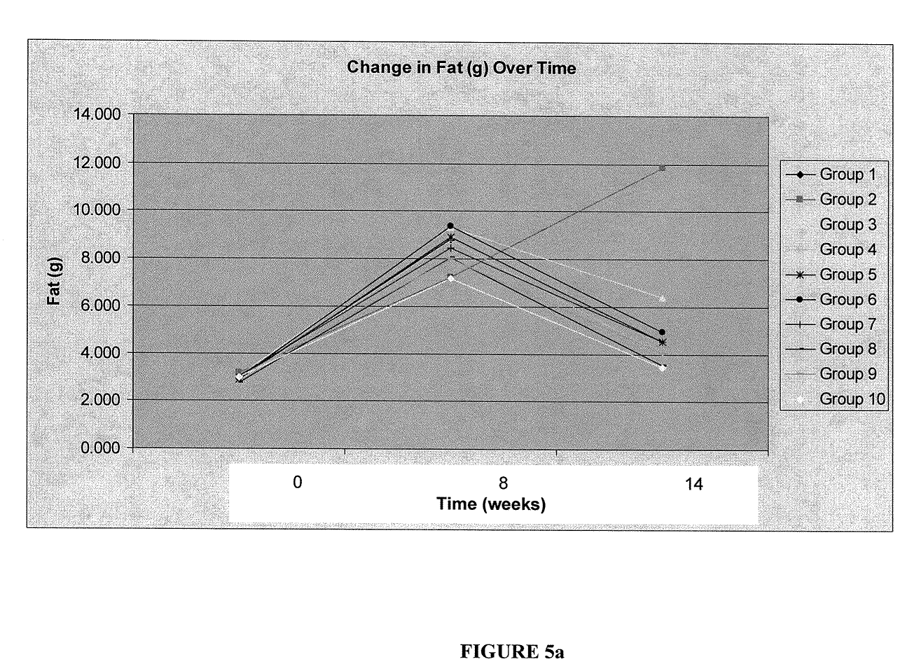

FIG. 4 is a graph showing the ratio of lean tissue mass to fat mass for Groups 2, 3, 4 and 10 at the end of the study. Each of the displayed treatment groups had a statistically significant different lean-to-fat ratio. Treatment Group 10, which was fed a diet having about 30% protein, wherein all of the protein was from milk-derived protein, exhibited the highest ratio of lean-to-fat, at about 6. Groups 2, 3, and 4 exhibited ratios of about 2.25, 3.5 and 5.25, respectively. FIGS. 5a and 5b show the change in lean tissue mass and fat mass, respectively.

3. Bone Mass

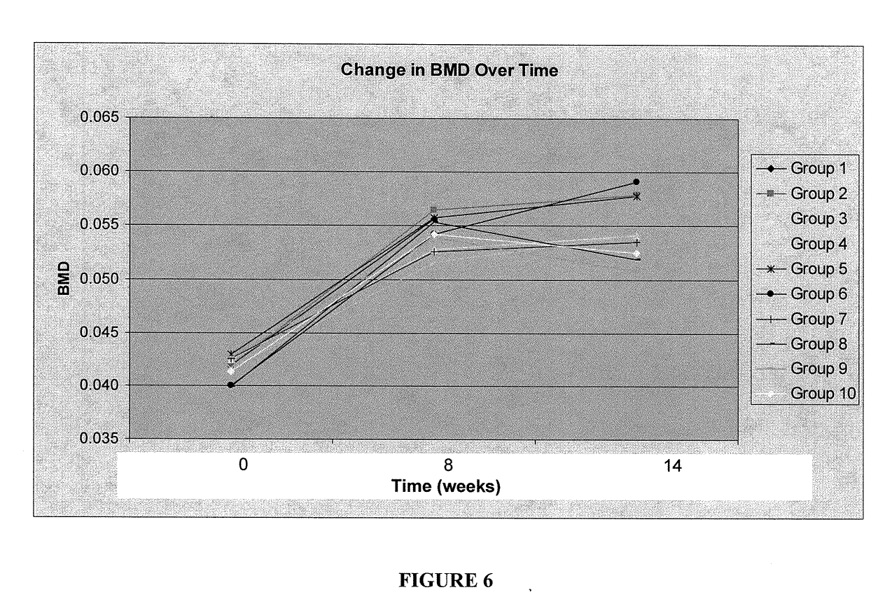

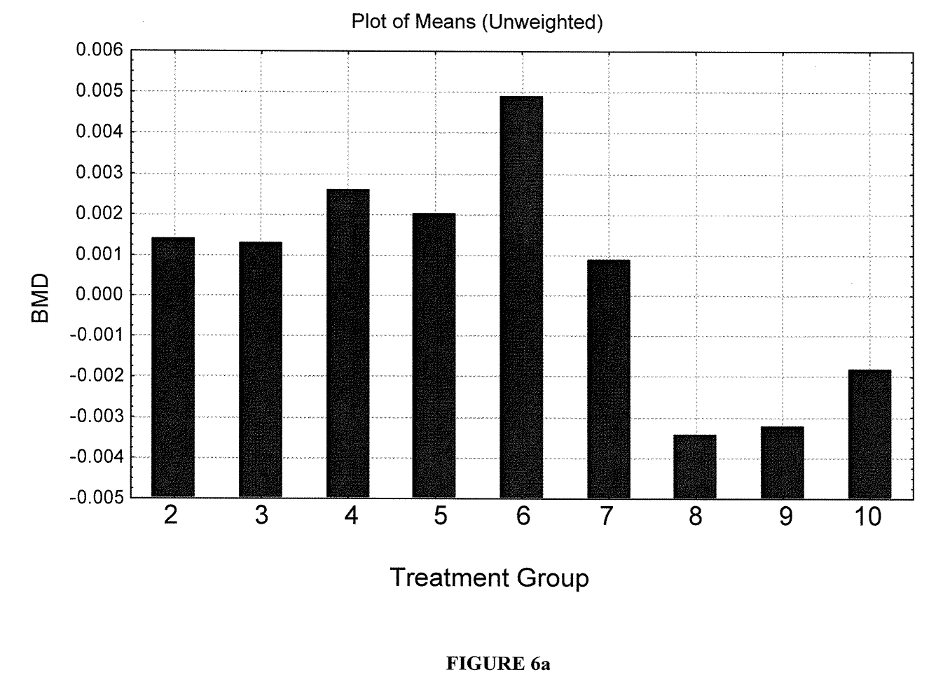

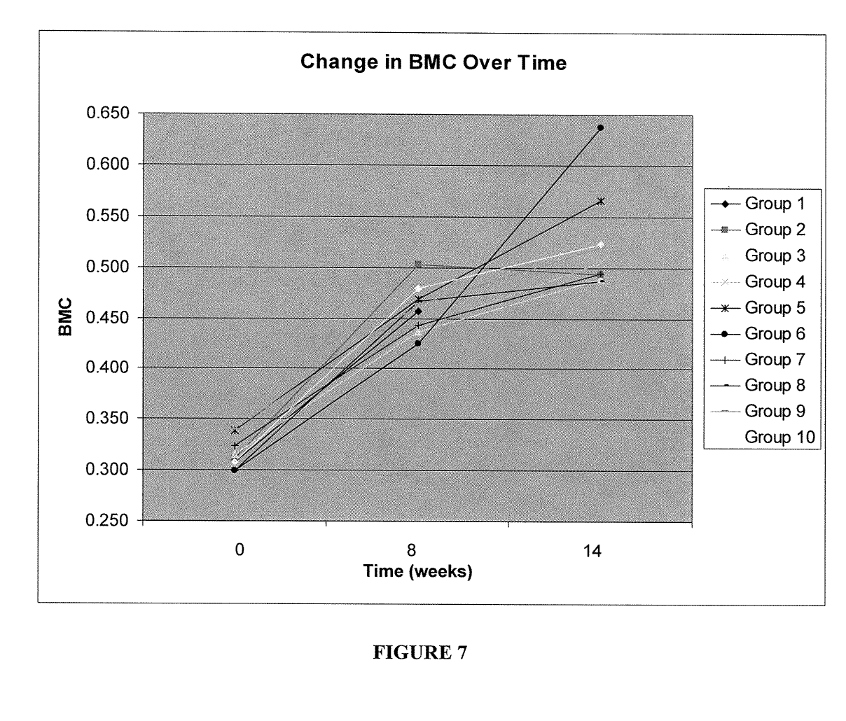

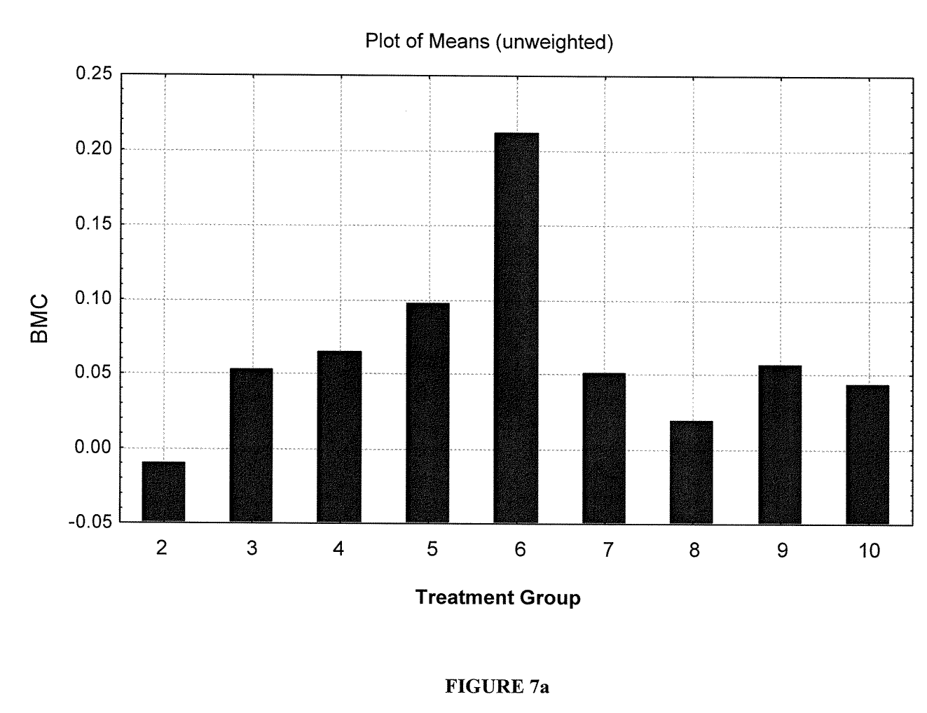

FIGS. 6 and 7 show the change in bone mineral density (BMD) and bone mineral content (BMC), respectively, for each of the treatment groups. FIGS. 6a and 7a show mean BMD and BMC, respectively, for each of the treatment groups at the end of the study. Group 6, which was fed a diet high in milk minerals, including calcium, exhibited significantly higher BMD and BMC as compared to the other treatment groups. This occurred despite the mice being on a reduced calorie diet.

While particular embodiments of the present invention have been described and illustrated, it should be understood that the invention is not limited thereto since modifications may be made by persons skilled in the art. The present application contemplates any and all modifications that fall within the spirit and scope of the underlying invention disclosed and claimed herein. For example, one skilled in the art will readily realize that a diet may be formulated that incorporates peptides, either derived from milk or from other suitable sources, and intact protein, in the form of protein isolates derived from milk or from other suitable sources. Similarly, these and other diets within the scope of the invention may be fortified with varying amounts of calcium and be implemented in calorically-restrictive regimens without departing from the scope of the invention.

What is claimed is:

1. A dietary supplement for treating a body weight condition comprising:
a milk-derived enzyme-inhibiting peptide provided in a therapeutically effective amount for treating the body weight condition.

2. The supplement of claim 1, wherein the body weight condition is obesity.

3. The supplement of claim 1, wherein the milk-derived enzyme-inhibiting peptide is derived from an angiotensin converting enzyme hydrolysate.

4. The supplement of claim 1, wherein the enzyme-inhibiting peptide is angiotensin converting enzyme inhibiting peptide.

5. The supplement of claim 1, wherein the enzyme-inhibiting peptide is derived from milk or whey.

6. The supplement of claim 1 further comprising a milk-derived mineral component.

7. The supplement of claim 6, wherein the milk-derived mineral component includes one or more milk minerals selected from the group consisting of calcium, copper, magnesium, phosphorus, potassium, selenium and zinc.

8. The supplement of claim 6, wherein the milk-derived mineral component includes calcium.

9. The supplement of claim 8, which further maintains bone mineral density or bone mineral content in an individual.

10. The supplement of claim 1 further comprising a dairy protein component.

11. The supplement of claim 10, wherein the dairy protein component is derived from milk or whey.

12. The supplement of claim 10, wherein the dairy protein component includes one or more whey protein isolates.

13. The supplement of claim 10, wherein the dairy protein component includes κ-casein fragment 106-169.

14. A method of maintaining lean tissue mass in an individual comprising:
administering to the individual a dietary supplement including a milk-derived peptide component and a milk-derived protein component present in a therapeutically effective amounts for maintaining lean tissue mass.

15. The method of claim 14, wherein the lean tissue is lean muscle tissue.

16. The method of claim 14, wherein the milk-derived protein component includes one or more whey protein isolates.

17. The method of claim 14, wherein the ratio of lean tissue mass to fat mass in the individual is at least about 5.25.

18. The method of claim 14, wherein the ratio of lean tissue mass to fat mass in the individual is at least about 6.

19. A method of promoting weight loss in an individual comprising:
administering to the individual an enzyme-inhibiting peptide blend including one or more peptides derived from milk in a calorically restricted diet, wherein the one or more peptides are present in a therapeutically effective amount for promoting weight loss.

20. The method of claim 19, wherein the enzyme-inhibiting peptide blend includes angiotensin converting enzyme inhibiting peptide.
